(19) United States
(12) Patent Application Publication
(10) Pub. No.: US 2025/0074856 A1
(43) Pub. Date: Mar. 6, 2025

(54) POLYPHOSHATES AND POLYPHOSPHONATES AS RATE PROMOTERS FOR THE GLACIAL ACETIC ACID PROCESS

(71) Applicant: LyondellBasell Acetyls, LLC, Houston, TX (US)

(72) Inventors: Noel C. Hallinan, Loveland, OH (US); David L. Ramage, Friendswood, TX (US); Shane J. Weber, League City, TX (US); Ryan J. Mathews, Tomball, TX (US); Elizabeth I. Ross-Medgaarden, Houston, TX (US)

(73) Assignee: LyondellBasell Acetyls, LLC, Houston, TX (US)

(21) Appl. No.: 18/819,276

(22) Filed: Aug. 29, 2024

Related U.S. Application Data
(60) Provisional application No. 63/535,880, filed on Aug. 31, 2023.

(51) Int. Cl. C07C 51/12; C07C 51/09

(52) U.S. Cl. CPC C07C 51/12; C07C 51/09

(57) ABSTRACT

A process for producing and recovering acetic acid in an acetic acid production system is disclosed, the process comprising contacting methanol, methyl acetate, or a mixture of the two, and carbon monoxide in the presence of a reaction mixture comprising iodide under carbonylation conditions sufficient to form acetic acid. The reaction mixture comprises a carbonylation catalyst, water, and one or more promoters selected from the group consisting of Group I and Group II polyphosphate and polyphosphonate salts and mixtures thereof. An aspect of the process includes a method for reducing water in the acetic acid production process.

### CROSS-REFERENCE TO RELATED APPLICATIONS

The application claims the benefit of priority to U.S. Provisional Patent Application No. 63/535,880 filed on Aug. 31, 2023, which is incorporated herein by reference in its entirety.

### FIELD OF THE INVENTION

This disclosure relates to the production of glacial acetic acid (GAA). More particularly, the disclosure relates to rate promoter compounds in acetic acid production.

### BACKGROUND OF THE INVENTION

A carbonylation process of methanol is a desirable commercial route for synthesis of acetic acid. In such a process, methanol, methyl acetate, or any mixture of the two, and carbon monoxide are reacted in the liquid phase in the presence of a homogeneous catalyst such as a rhodium (Rh)-based catalyst, at for instance 150 to 200° C. and increased pressure to produce acetic acid with typically up to 95% selectivity and 5% side products. Methyl iodide (CH₃I, or MeI) is typically used as a promoter for the carbonylation step in this process. The commercial reaction proceeds in the liquid phase with a solvent such as methyl acetate using the homogeneous catalyst. A controlled amount of water required for the reaction is added and/or generated in situ by reaction of methanol with HI or by esterification of methanol with acetic acid. Given that water must be separated from the acetic acid in recovery, it is desirable to use lower amounts of water in the carbonylation process.

In the carbonylation process, the homogeneous catalyst is preferably cycled between oxidative states. For example, for a (Rh)-based catalyst, the catalytically active species may be the four-coordinate anion [Rh(CO)₂I₂]⁻, typically referred to as “Rh I”. The first organometallic step would be the oxidative addition of methyl iodide to [Rh(CO)₂I₂]⁻ to form the six-coordinate anion [(CH₃)Rh(CO)₂I₃]⁻. This anion rapidly transforms via the migration of a methyl group to an adjacent carbonyl (CO) ligand affording the five-coordinate acetyl anion [(CH₃CO)Rh(CO)I₃]⁻. This five-coordinate complex then reacts with carbon monoxide to form the six-coordinate dicarbonyl complex, which then undergoes reductive elimination to yield acetyl iodide (CH₃C(O)I). The catalytic cycle also involves two non-organometallic steps: conversion of methanol or methyl acetate to methyl iodide and the hydrolysis of acetyl iodide to acetic acid and hydrogen iodide. This reaction has been shown to be first-order with respect to methyl iodide and [Rh(CO)₂I₂]⁻. The first step of the catalytic cycle is oxidative addition of methyl iodide.

It would be desirable to improve this carbonylation process by accelerating or “promoting” the rate of formation of acetic acid, such as by promoting the rate of methyl iodide oxidative addition. Methods that could suppress the undesirable oxidative addition of HI with [Rh(CO)₂I₂]⁻ and support a reduced amount of water in the process would be additionally desirable.

In particular, the two oxidative addition reactions that control the pathways leading to desired methanol carbonylation (A) and undesired water gas shift (WGS) (B) are as shown below:

MeI+Rh I→GAA (A)

HI+Rh I→H₂+CO₂ (B)

As such, any new GAA production method would desirably be capable of achieving acceleration of MeI oxidative addition and/or suppression of HI oxidative addition. Desirably the optimal production method will both accelerate MeI oxidative addition and suppress HI oxidative addition to Rh I. Suppression of HI oxidative addition to Rh I will free up additional Rh I for MeI oxidative addition.

### SUMMARY OF THE INVENTION

A process for producing and recovering acetic acid in an acetic acid production system is disclosed, the process comprising contacting methanol and/or methyl acetate and carbon monoxide in the presence of a reaction mixture comprising iodide under carbonylation conditions sufficient to form acetic acid. The reaction mixture comprises a carbonylation catalyst, water, and one or more rate promoting compounds (“promoters”) selected from the group consisting of Group I and Group II polyphosphate and polyphosphonate salts and mixtures thereof.

Embodiments of the disclosure include processes for producing acetic acid comprising (or consisting of, or consisting essentially of):
a) combining methanol, methyl acetate or any mixture of the two, and carbon monoxide in the presence of a reaction mixture comprising:
   i) a carbonylation catalyst selected from the group consisting of rhodium catalysts, iridium catalysts, palladium catalysts, and cobalt catalysts;
   ii) water in the range of from 0.1 wt % to 10 wt %, based on the weight of the reaction mixture; and
   iii) one or more promoters selected from the group consisting of Group I and Group II polyphosphate and polyphosphonate salts and mixtures thereof; and
   iv) iodide; and
b) recovering acetic acid.

Embodiments of the disclosure also include methods for reducing water in an acetic acid production process comprising (or consisting of, or consisting essentially of):
a) combining methanol, methyl acetate or any mixture of the two, and carbon monoxide in the presence of a reaction mixture comprising iodide under carbonylation conditions sufficient to form acetic acid at a first rate, wherein the reaction mixture comprises:
   i) a carbonylation catalyst, selected from the group consisting of rhodium catalysts, iridium catalysts, palladium catalysts, and cobalt catalysts; and
   ii) a first amount of water, where the water is present in the range of from 0.1 wt % to 10 wt %, based on the weight of the reaction mixture; and
b) combining one or more promoters to the reaction mixture at an iodide to promoter molar ratio of greater than 2, wherein the one or more promoters are selected from the group consisting of Group I and Group II polyphosphate and polyphosphonate salts and mixtures thereof; and
c) reducing the wt % of water in the reaction mixture to a second amount of water while maintaining a second rate of acetic acid production that is the same as or greater than the first rate of acetic acid production.

While the disclosed processes are susceptible to various modifications and alternative forms, the drawings illustrate specific embodiments herein described in detail by way of example. It should be understood, however, that the description herein of specific embodiments is not intended to limit the invention to the particular embodiments disclosed, but on the contrary, the intention is to cover all modifications, equivalents, and alternatives falling within the spirit and scope of the invention as defined by the appended claims.

### BRIEF DESCRIPTION OF THE DRAWINGS

The claimed subject matter may be understood by reference to the following description taken in conjunction with the accompanying drawings, in which like reference numerals identify like elements, and in which:

FIG. 1 is a schematic of an exemplary acetic acid production system in accordance with embodiments and/or techniques disclosed herein;

FIG. 2 is a graphical representation of FTIR overlay spectra of 0.04M DTPMP-Na₇ combined with 0.015M Rh I, no added MeI from 0 to 3 hours;

FIG. 3 is a graphical representation of FTIR overlay spectra of 0.0-0.11M DTPMP-Na₇ (1.1-2.3 H₂O M) combined with 0.015M Rh I;

FIG. 4 is a graphical representation of FTIR overlay spectra of 0.01M HI combined with variable DTPMP-Na₇ concentrations with 0.015M Rh I from 0 to 4 hours;

FIG. 5 is a graphical representation of FTIR overlay spectra of 2M MeI combined with variable DTPMP-Na₇ concentrations with 0.015M Rh I from 0 to 3 hours;

FIG. 6 is a plot of rate constant as a function of IP₆-Na₁₂ concentration or IP₆ concentration for MeI oxidative addition reactions;

FIG. 7 is a plot of Rh I catalyst concentration as a function of time for various concentrations of IP₆-Na₁₂ promoter for HI oxidative addition;

FIG. 8 is a plot of Rh I catalyst concentration as a function of time for various concentrations of IP₆ acid for HI oxidative addition;

FIG. 9 is a plot of relative rate constants of LiOAc, and the novel promoters IP₆-Na₁₂ promoter and DTPMP-Na₇;

FIG. 10 is a graphical representation of FTIR overlay spectra of 0.1M HI oxidative addition to 0.015M Rh I with variable LiI concentrations;

FIG. 11 is a plot of Rh I catalyst concentration as a function of time for various concentrations of LiI concentrations for HI oxidative addition;

FIG. 12 is a graphical representation of FTIR overlay spectra of 0.1M HI oxidative addition to Rh I with variable LiOAc/LiI; and

FIG. 13 is a graphical representation of FTIR overlay spectra of 2M MeI oxidative addition to Rh I.

### DETAILED DESCRIPTION OF THE INVENTION

The designation of groups of the Periodic Table of the Elements as used herein is in accordance with the current IUPAC convention. The expression “MeI” is used herein as an abbreviation for methyl iodide. The expression “HI” is used herein as an abbreviation for hydrogen iodide. The expression “acac” is used herein as an abbreviation for acetoacetate anion, i.e., H₃CC(═O)CH₂C(═O)O—. Unless specifically indicated otherwise, the expression “wt %” as used herein refers to the percentage by weight of a particular component in the referenced composition. The expression “LiOAc” is an abbreviation for lithium acetate.

The term “alkyl” means a monovalent group of carbon and hydrogen (such as a C₁ to C₃₀, such as a C₁ to C₁₂ group). Alkyl groups in a compound are typically bonded to the compound directly via a carbon atom. Unless otherwise specified, alkyl groups may be linear (i.e., unbranched) or branched, be cyclic, acyclic, or part cyclic/acyclic. In an embodiment the alkyl group comprises a linear or branched acyclic alkyl group. Representative examples of alkyl groups include, but are not limited to, methyl, ethyl, n-propyl, iso-propyl, n-butyl, sec-butyl, iso-butyl, tert-butyl, n-pentyl, iso-pentyl, neo-pentyl, hexyl, heptyl, octyl, dimethyl hexyl, nonyl, decyl, undecyl, dodecyl, tridecyl, tetradecyl, pentadecyl, hexadecyl, heptadecyl, octadecyl, nonadecyl, icosyl and triacontyl.

The term “alkylene” means a divalent group of carbon and hydrogen (such as a C₁ to C₃₀, such as a C₁ to C₁₂ group).

Embodiments of the disclosed process and system involve the production of acetic acid by carbonylating methanol in a carbonylation reaction. The carbonylation reaction may be represented by: CH₃OH+CO→CH₃COOH.

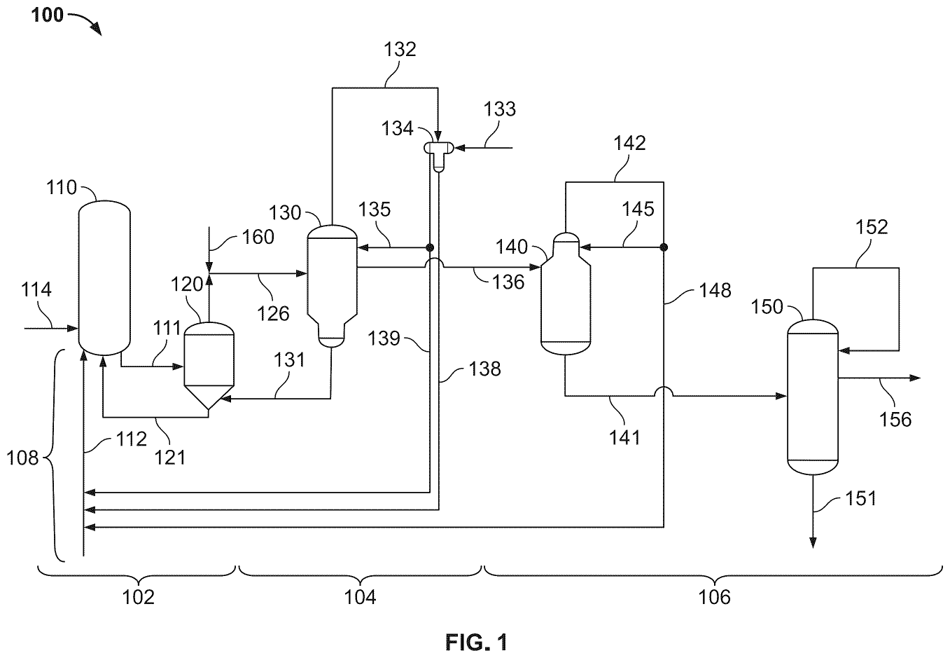

FIG. 1 is a schematic of an exemplary acetic acid production system 100 implementing the carbonylation reaction. In some embodiments, the acetic acid system 100 may include a reaction area 102, a light-ends area 104, a purification area 106, and recycle area 108. The reaction area 102 may include a reactor 110, a flash vessel 120, and associated equipment. The reactor 110 is a reactor or vessel in which methanol is carbonylated in the presence of a catalyst to form acetic acid at elevated pressure and temperature. Note that the “streams” discussed herein may be part of more than one functional arca. A “reaction mixture” is present in the reactor 110, from which a portion may be withdrawn, and to which components (such as catalysts, chemical reactants, promoters, diluent, etc.) may be introduced, combined and mixed to obtain a desirable concentration; and reacted and/or maintained at a desirable concentration.

The reaction arca 102 may comprise a reactor 110, a flash vessel 120, equipment associated with the reactor 110 and flash vessel 120, and streams associated with the reactor 110 and flash vessel 120. For example, the reaction area 102 may comprise reactor 110, flash vessel 120, and streams (or portions of streams) 111, 112, 114, 121, 126, 131, 160, 138, 139, 148. The reactor 110 is a reactor or vessel in which methanol is carbonylated in the presence of a catalyst to form acetic acid at elevated pressure and temperature. The flash vessel 120 is a tank or vessel in which a reaction mixture obtained in the reactor, for example the reactor 110, is at least partially depressurized and/or cooled to form a vapor stream and a liquid stream. A vapor stream is a product or composition which comprises components in the gaseous state under the conditions of the processing step in which the stream is formed. A liquid stream may be a product or composition which comprises components in the liquid state under the conditions of the processing step in which the stream is formed.

The light-ends area 104 may comprise a separations column, for example a light-ends column 130, equipment associated with light-ends column 130, and streams associated with the light-ends column 130. For example, the light-ends area 104 may comprise light-ends column 130, decanter 134, and streams 126, 131, 132, 133, 135, 136, 138, 139, 160. The light-ends column 130 is a fractioning or distillation column and includes any equipment associated with the column, including but not limited to heat exchangers, decanters, pumps, compressors, valves, and the like.

The purification area 106 may comprise a drying column 140, optionally, a heavy-ends column 150, equipment associated with drying column 140 and heavy-ends column 150, and streams associated with the drying column 140 and heavy-ends column 150. For example, the purification arca 106 may comprise drying column 140, heavy-ends column 150, and streams 136, 141, 142, 145, 148, 151, 152, 156. The heavy-ends column 150 is a fractioning or distillation column and includes any equipment associated with the column, including but not limited to heat exchangers, decanters, pumps, compressors, valves, and the like.

The recycle area 108 may comprise process streams recycled to the reaction arca 102 and/or light-ends area 104. For example, in FIG. 1, the recycle area 108 may comprise streams 121, 138, 139, 148.

In an embodiment, the reactor 110 may be configured to receive a carbon monoxide feed stream 114 and a methanol feed stream 112. The feed stream 112 may comprise a methanol feed stream, a methyl acetate feed stream or any mixture of the two. In the illustrated embodiment, the feed stream 112 is a mixed stream. A reaction mixture may be withdrawn from the reactor in stream 111. Other streams may be included such as, for example, a stream that may recycle a bottoms mixture of the reactor 110 back into the reactor 110, or a stream may be included to release a gas from the reactor 110. Stream 111 may comprise at least a part of the reaction mixture.

In an embodiment, the flash vessel 120 may be configured to receive stream 111 from the reactor 110. In the flash vessel 120, stream 111 may be separated into a vapor stream 126 and a liquid stream 121. The vapor stream 126 may be communicated to the light-ends column 130, and the liquid stream 121 may be communicated to the reactor 110. In an embodiment, stream 126 may have acetic acid, water, methyl iodide, methyl acetate, HI, mixtures thereof.

In an embodiment, the light-ends column 130 may be a distillation column and associated equipment such as a decanter 134, pumps, compressors, valves, and other related equipment. The light-ends column 130 may be configured to receive stream 126 from the flash vessel 120. In the illustrated embodiment, stream 132 is the overhead product from the light-ends column 130, and stream 131 is bottoms product from the light-ends column 130. As indicated, light-ends column 130 may include a decanter 134, and stream 132 may pass into decanter 134.

Stream 135 may emit from decanter 134 and recycle back to the light-ends column 130. Stream 138 may emit from decanter 134 and may recycle back to the reactor 110 via, for example, stream 112 or be combined with any of the other streams that feed the reactor. Stream 139 may recycle a portion of the light phase of decanter 134 back to the reactor 110 via, for example, stream 112. Stream 136 may emit from the light-ends column 130. Other streams may be included such as, for example, a stream that may recycle a bottoms mixture of the light-ends column 130 back into the light-ends column 130. Streams received by or emitted from the light-ends column 130 may pass through a pump, compressor, heat exchanger, and the like as is common in the art.

In an embodiment, the drying column 140 may be a vessel and associated equipment such as heat exchangers, decanters, pumps, compressors, valves, and the like. The drying column 140 may be configured to receive stream 136 from the light-ends column 130. The drying column 140 may separate components of stream 136 into streams 142 and 141.

Stream 142 may emit from the drying column 140, recycle back to the drying column via stream 145, and/or recycle back to the reactor 110 through stream 148 (via, for example, stream 112). Stream 141 may emit from the drying column 140 and may include de-watered crude acetic acid product. Stream 142 may pass through equipment such as, for example, a heat exchanger or separation vessel before streams 145 or 148 recycle components of stream 142. Other streams may be included such as, for example, a stream may recycle a bottoms mixture of the drying column 140 back into the drying column 140. Streams received by or emitted from the drying column 140 may pass through a pump, compressor, heat exchanger, separation vessel, and the like as is common in the art.

The heavy-ends column 150 may be a distillation column and associated equipment such as heat exchangers, decanters, pumps, compressors, valves, and the like. The heavy-ends column 150 may be configured to receive stream 141 from the drying column 140. The heavy-ends column 150 may separate components from stream 141 into streams 151, 152, and 156. Streams 151 and 152 may be sent to additional processing equipment (not shown) for further processing. Stream 152 may also be recycled, for example, to light-ends column 130. Stream 156 may have acetic acid product.

A single column (not depicted) may be used in the place of the combination of the light-ends distillation column 130 and the drying column 140. The single column may vary in the diameter/height ratio and the number of stages according to the composition of vapor stream from the flash separation and the requisite product quality. For instance, U.S. Pat. No. 5,416,237, the teachings of which are incorporated herein by reference, discloses a single column distillation.

Alternative embodiments for the acetic acid production system 100 may also be found in U.S. Pat. Nos. 6,552,221, 7,524,988, and 8,076,512, which are fully incorporated herein by reference.

In an embodiment, the carbonylation reaction in reactor 110 of system 100 may be performed in the presence of a carbonylation catalyst. Catalysts may include, for example, rhodium catalysts and iridium catalysts.

Suitable rhodium catalysts are taught, for example, by U.S. Pat. No. 5,817,869, which is herein incorporated by reference. The rhodium catalysts may include rhodium metal and rhodium compounds. In an embodiment, the rhodium compounds may be selected from the group consisting of rhodium salts, rhodium oxides, rhodium acetates, organo-rhodium compounds, coordination compounds of rhodium, the like, and mixtures thereof. In an embodiment, the rhodium compounds may be selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. In an embodiment, the rhodium compounds may be selected from the group consisting of [H]Rh(CO)₂I₂, Rh(CH₃CO₂)₂, the like, and mixtures thereof.

Suitable iridium catalysts are taught, for example, by U.S. Pat. No. 5,932,764, which is herein incorporated by reference. The iridium catalysts may include iridium metal and iridium compounds. Examples of suitable iridium compounds include IrCl₃, IrI₃, IrBr₃, [Ir(CO)₂I]₂, [Ir(CO)₂Cl]₂, [Ir(CO)₂Br]₂, [Ir(CO)₄I₂]—H+, [Ir(CO)₂Br₂]—H+, [IR(CO)₂I₂]—H+, [Ir(CH₃)I₃(CO)₂]—H+, Ir₄(CO)1₂, IrCl₃.4H₂O, IrBr₃.4H₂O, Ir₃(CO)1₂, Ir₂O₃, IrO₂, Ir(acac)(CO)₂, Ir(acac)₃, Ir(OAc)₃, [Ir₃O(OAc)₆(H₂O)₃][OAc], H₂[IrCl₆], the like, and mixtures thereof. In an embodiment, the iridium compounds may be selected from the group consisting of acetates, oxalates, acetoacetates, the like, and mixtures thereof. In an embodiment, the iridium compounds may be one or more acetates.

In an embodiment, the catalyst may be used with a co-catalyst. In an embodiment, co-catalysts may include metals and metal compounds selected from the group consisting of osmium, rhenium, ruthenium, cadmium, mercury, zinc, gallium, indium, and tungsten, their compounds, the like, and mixtures thereof. In an embodiment, co-catalysts may be selected from the group consisting of ruthenium compounds and osmium compounds. In an embodiment, co-catalysts may be one or more ruthenium compounds. In an embodiment, the co-catalysts may be one or more acetates.

The reaction rate depends upon the concentration of the carbonylation catalyst in the reaction mixture in reactor 110. In some embodiments the catalyst concentration is at least 2 mmol/l, or at least 3 mmol/l, or at least 5 mmol/l. In some embodiments the catalyst concentration is at most 150 mmol/l, or at most 100 mmol/l, or at most 25 mmol/l. In particular embodiments the catalyst concentration is from about 2 to about 150 mmol/l, or from about 3 to about 100 mmol/l, or from about 5 to about 25 mmol/l.

In an embodiment, the carbonylation reaction in reactor 110 of system 100 may be performed in the presence of a catalyst stabilizer. Suitable catalyst stabilizers include at least two types of catalyst stabilizers. The first type of catalyst stabilizer may be a metal iodide salt such as lithium iodide. The second type of catalyst stabilizer may be a non-salt stabilizer. In an embodiment, non-salt stabilizers may be pentavalent Group 15 oxides, such as that disclosed in U.S. Pat. No. 9,790,159, which is herein incorporated by reference. The pentavalent Group 15 oxide is soluble in acetic acid and in an embodiment comprises a bidentate phosphine dioxide, tertiary arsine oxides, the like, or combinations thereof. Nonlimiting examples of suitable bidentate phosphine dioxide for use as a pentavalent Group 15 oxide include, but are not limited to, bis(diphenylphosphino)methane dioxide (bis-DPPMeO₂), bis(diphenylphosphino)propane dioxide (bis-DPPPrO₂), bis(diphenylphosphino)pentane dioxide (bis-DPPPeO₂), and combinations thereof. Nonlimiting examples of suitable phosphine oxides for use as a pentavalent Group 15 oxide include, but are not limited to, triphenyl arsine oxide (TPAsO), triethylarsine oxide (TEtAsO), and combinations thereof. In an embodiment, the catalyst stabilizer may be one or more phosphine oxides.

In an embodiment, hydrogen may also be fed into the reactor 110. Addition of hydrogen can enhance the carbonylation efficiency. In an embodiment, the concentration of hydrogen may be in a range of from about 0.1 mol % to about 5 mol % of carbon monoxide in the reactor 110. In an embodiment, the concentration of hydrogen may be in a range of from about 0.3 mol % to about 3 mol % of carbon monoxide in the reactor 110.

In an embodiment, the carbonylation reaction in reactor 110 of system 100 may be performed in the presence of water. In an embodiment, the concentration of water is from about 0.1 wt % to about 10.0 wt %, about 0.2 wt % to about 6.0 wt %, about 0.3 wt % to about 4.5 wt %, or about 0.4 wt % to about 2 wt %, based on the total weight of the reaction mixture.

In an embodiment, the carbonylation reaction may be performed in the presence of methyl acetate. Methyl acetate may be formed in situ. In embodiments, methyl acetate may be added as a starting material to the reaction mixture. In an embodiment, the concentration of methyl acetate may be from about 2 wt % to about 20 wt % based on the total weight of the reaction mixture. In an embodiment, the concentration of methyl acetate may be from about 2 wt % to about 16 wt %. In an embodiment, the concentration of methyl acetate may be from about 2 wt % to about 8 wt %. Alternatively, methyl acetate or a mixture of methyl acetate and methanol from byproduct streams of the methanolysis of polyvinyl acetate or ethylene-vinyl acetate copolymers can be used for the carbonylation reaction.

In an embodiment, the carbonylation reaction may be performed in the presence of methyl iodide that is added to the reactor 110, generated in the reactor, or both. In an embodiment, the concentration of MeI may be from about 0.6 wt % to about 36 wt % based on the total weight of the reaction mixture. In an embodiment, the concentration of MeI may be from about 4 wt % to about 24 wt %. In an embodiment, the concentration of MeI may be from about 6 wt % to about 20 wt %. Alternatively, MeI may be generated in the reactor 110 by adding HI.

As mentioned, methanol and carbon monoxide may be fed to the reactor 110 in stream 112 and stream 114, respectively. The methanol feed stream to the reactor 110 may come from a syngas-methanol facility or any other source. Methanol does not react directly with carbon monoxide to form acetic acid. It is converted to MeI by the HI present in the reactor 110 and then reacts with carbon monoxide and water to give acetic acid and regenerate the HI. Therefore, in any embodiment the concentration of HI present in the reactor is from about 0.1 wt % to about 10.0 wt %, about 0.2 wt % to about 6.0 wt %, about 0.3 wt % to about 4.5 wt %, or about 0.4 wt % to about 2 wt %, based on the total weight of the reaction mixture.

In any embodiment MeI reacts with the carbonylation catalyst in a rate limiting step in the acidic acid process to form an active catalyst species. While HI may be added to the reaction mixture to generate MeI, it is desirable to modulate the presence of the HI as it is believed to be a carbonylation catalyst inhibitor. Thus, in any embodiment a promoter compound is added to the reaction mixture in amounts that are beneficial to both ends.

In any embodiment one or more promoters are combined and maintained in the reaction mixture, in any part of the acetic acid production system 100, preferably upstream of the purification area 106, wherein the one or more promoters are selected from the group consisting of Group I and Group II polyphosphate and polyphosphonate salts and mixtures thereof.

In any embodiment, the polyphosphate salts comprise 2, or 3 or more phosphate [(PO₄)²⁻] groups, wherein an oxygen atom on each phosphorus is directly bound to a carbon atom comprising an R group; wherein the R group can be a linear, branched, or cyclic alkyl or alkylene group. In any embodiment the polyphosphate salts comprise 2, or 3 to 5, or 8, or 10 phosphate groups. In yet another embodiment the polyphosphate salts comprise aminopolyphosphate salts comprising at least one amine group and at least 2, 3 or 4 phosphate groups. In yet another embodiment the polyphosphate salts comprise aminopolyphosphate salts comprising at least 2 amine groups and at least 2, 3, or 4 phosphate groups. In any embodiment the R group comprises one or more alkylamine or alkylene amine groups, which can also comprise one or more phosphate or phosphonate groups to comprise the overall polyphosphate salt or polyphosphonate salt.

In any embodiment, the polyphosphonate salts comprise 2, or 3 or more phosphonate [(PO₃)²⁻] groups, wherein each phosphorous is directly bound to a carbon atom comprising an R group; wherein the R group can be a linear, branched, or cyclic alkyl or alkylene group. In any embodiment the polyphosphonate salts comprise 2, or 3 to 5, or 8, or 10 phosphonate groups. In yet another embodiment the polyphosphonate salts comprise aminopolyphosphonate salts comprising at least one amine group and at least 2, 3 or 4 phosphonate groups. In yet another embodiment the polyphosphate salts comprise aminopolyphosphonate salts comprising at least 2 amine groups and at least 2, 3, or 4 phosphonate groups. In any embodiment the R group comprises one or more alkylamine or alkylene amine groups, which can also comprise one or more phosphate or phosphonate groups to comprise the overall polyphosphate salt or polyphosphonate salt.

Stated alternately, in any embodiment the polyphosphate and polyphosphonate salts described herein may be represented by the general formula representing the anionic portion [(PO₄)QRₙ)⁻²]ₓ and [(PO₃)QR′)⁻²]ₓ, respectively, wherein a promoter compound may comprise “x” number of units and corresponding Group I and/or II cations, wherein x is 2, 3, 4 or more; and wherein each R group can be the same or different and can be a linear, branched, or cyclic alkyl or alkylene group; and wherein Q may be a carbon or nitrogen atom and “n” is 2 or 3 and may be bonded further to an R; and wherein when at least one R is an alkylene group that may be bound to another [(PO₄)QRₙ)⁻²]ₓ or [(PO₃)QR′)⁻²]ₓ, unit, preferably “x” number of units.

In any embodiment, the Group I and Group II polyphosphate and polyphosphonate salts may be completely ionized to form complete salts, or may be partially ionized as a mixture of the salt (e.g., Na⁺ or Ca²⁺) and acid (H+).

Non-limiting examples of promoters are polyalkylencaminephosphonate salts, such as diethylenetriaminepentamethylenephosphonate hepta sodium salt (DTPMPA-Na₇) (structure 1 below, complete acid structure shown, H+ instead of Group I or II cations) and other similar compounds comprising 2 or more phosphate and/or phosphonate groups and at least one amine group, preferably two or more alkylencamine groups with 2 or more phosphate and/or phosphonate groups attached thereto; and polyalkylenephosphate salts or polyalkylenephosphonate salts such as inositol hexakisphosphate sodium dodecanoate, (IP₆-Na₁₂) (structure 2 below) and other similar compounds comprising 2 or more phosphate and/or phosphonate groups; where various molar equivalents of water of hydration may be present. Other alkyl, phosphate, and phosphonate substituted versions of these, with any combination of Group I and Group II cations, are possible promoters as disclosed herein.

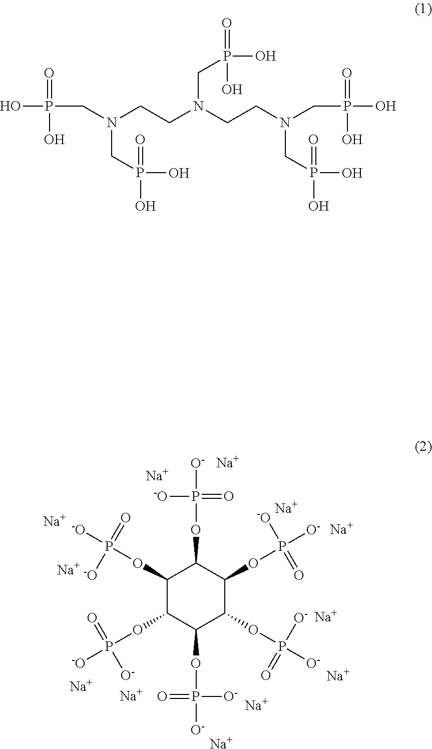

In any embodiment, salts of glutamate (C₅H₈O₄N) and dicarboxymethyl alaninate (C₇H₈NO₆) are absent from the reaction mixture, meaning that they are not added to the reaction mixture and desirably absent (0 ppm), or if present, are present in an amount of less than 0.2 ppm, or 0.1 ppm. However, substituted versions of glutamate may be present as the one or more promoters as described herein such as tetrasodium N,N-Bis(carboxymethyl)-L-glutamate (GLDA-Na₄).

Thus in any embodiment is a process for producing acetic acid comprising (or consisting of, or consisting essentially of): a) combining methanol and carbon monoxide in the presence of a reaction mixture comprising: i) a carbonylation catalyst selected from the group consisting of rhodium catalysts, iridium catalysts, palladium catalysts, and cobalt catalysts; ii) water in the range of from 0.1 wt % to 10 wt %, based on the weight of the reaction mixture; and iii) one or more promoters selected from the group consisting of Group I and Group II polyphosphate and polyphosphonate salts and mixtures thereof; and iv) iodide; and b) recovering acetic acid.

Also in any embodiment is a method for reducing water in an acetic acid production process comprising (or consisting of, or consisting essentially of): a) combining methanol and carbon monoxide in the presence of a reaction mixture comprising iodide under carbonylation conditions sufficient to form acetic acid at a first rate, wherein the reaction mixture comprises: i) a carbonylation catalyst, selected from the group consisting of rhodium catalysts, iridium catalysts, palladium catalysts, and cobalt catalysts; and ii) a first amount of water, where the water is present in the range of from 0.1 wt % to 10 wt %, based on the weight of the reaction mixture; and b) combining one or more promoters to the reaction mixture at an iodide to promoter ratio of greater than 2, wherein the one or more promoters are selected from the group consisting of Group I and Group II polyphosphate and polyphosphonate salts and mixtures thereof; and c) reducing the wt % of water in the reaction mixture to a second amount of water while maintaining a second rate of acetic acid production that is the same as or greater than the first rate of acetic acid production.

In any embodiment the amount of promoter maintained in the reaction mixture is such that a molar ratio to carbonylation catalyst is greater than about 0.5:1. In some embodiments, the molar ratio of the promoter to rhodium is from about 0.5:1 to about 100:1. In some embodiments, from about 0.005 to about 2.0 M of the promoter may be in the reaction mixture. In some embodiments, from about 0.01 to about 1.5 M, or from 0.025 to 1.2 M, of the promoter may be in the reaction mixture.

In any embodiment the amount of promoter maintained in the reaction mixture is such that a molar ratio of iodide to promoter is greater than 2:1, or greater than 3:1, or greater than 4:1, or greater than 6:1. In some embodiments, the molar ratio of the iodide to promoter is from 2:1 to 40:1, or from 3:1 to 35:1, or from 4:1 to 30:1, or from 6:1 to 25:1. Also in any embodiment the one or more promoters are maintained at a promoter to carbonylation catalyst molar ratio of 0.005:1 to 4:1, or from 0.01:1 to 3:1, or from 0.02:1 to 2:1.

In an embodiment, the rate at which promoter is introduced into the system 100 may be adjusted depending on the water, carbonylation catalyst, and/or HI content. In some embodiments, the promoter may be maintained in an amount of at least about 0.1 mol per mol HI. In some embodiments, at least about 0.5 mol promoter, or at least about 1 mol promoter, or at least about 1.5 mol promoter, per mol HI is maintained. In some embodiments, the promoter may be maintained in an amount of from about 0.1 to about 10 mol per mol HI. In some embodiments, the amount of promoter is from about 0.25 to about 7.5 mol, or from about 0.5 to about 5 mol, or from about 0.75 to about 1.5 mol, per mol HI.

In other embodiments, the amount of promoter is from about 1 to about 10 mol, or from about 1 to about 7.5 mol, or from about 1 to about 5 mol, per mol HI. In some embodiments, the promoter may be maintained in an amount from about 0.1 to about 1.5 mol per mol HI. In some embodiments, the amount of promoter maintained may be from about 0.1 to about 1.3 mol, or from about 0.1 to about 1.1 mol, per mol HI. In further some embodiments, the amount of promoter is from about 0.5 to about 3 mol, or from about 0.5 to about 2 mol, or from about 0.5 to about 1.5 mol, per mol HI.

In an embodiment, the carbonylation reaction in reactor 110 of system 100 may occur at a temperature within the range of about 120° C. to about 250° C., alternatively, about 150° C. to about 250° C., alternatively, about 150° C. to about 200° C. In an embodiment, the carbonylation reaction in reactor 110 of system 100 may be performed under a pressure within the range of about 200 psia (1.38 MPa-a) to 2,000 psia (13.8 MPa-a), alternatively, about 200 psia (1.38 MPa-a) to about 1,000 psia (6.9 MPa-a), alternatively, about 300 psia (2.1 MPa-a) to about 500 psia (3.4 MPa-a).

In an embodiment, the reaction mixture may be withdrawn from the reactor 110 through stream 111 and is flashed in flash vessel 120 to form a vapor stream 126 and a liquid stream 121. The reaction mixture in stream 111 may include acetic acid, methanol, methyl acetate, methyl iodide, carbon monoxide, carbon dioxide, water, HI, heavy impurities, catalyst, or combinations thereof. The flash vessel 120 may comprise any configuration for separating vapor and liquid components via a reduction in pressure. For example, the flash vessel 120 may comprise a flash tank, nozzle, valve, or combinations thereof.

The flash vessel 120 may have a pressure below that of the reactor 110. In an embodiment, the flash vessel 120 may have a pressure of from about 10 psig (69 kPa-g) to 100 psig (689 kPa-g). In an embodiment, the flash vessel 120 may have a temperature of from about 100° C. to 160° C.

The vapor stream 126 may include acetic acid and other volatile components such as methanol, methyl acetate, methyl iodide, carbon monoxide, carbon dioxide, water, entrained HI, complexed HI, and mixtures thereof. The liquid stream 121 may include acetic acid, methanol, methyl acetate, methyl iodide, carbon monoxide, carbon dioxide, water, complexed HI, HI, an azcotrope of HI and water, and mixtures thereof. In particular, the liquid stream may comprise the catalyst, complexed HI, HI, an azcotrope of HI and water, and mixtures thereof. The liquid stream 121 may further comprise sufficient amounts of water and acetic acid to carry and stabilize the catalyst, non-volatile catalyst stabilizers, or combinations thereof. The liquid stream 121 may recycle to the reactor 110. The vapor stream 126 may be communicated to light-ends column 130 for distillation.

In an embodiment, the vapor stream 126 may be distilled in a light-ends column 130 to form an overhead stream 132, a crude acetic acid product stream 136, and a bottoms stream 131. In an embodiment, the light-ends column 130 may have at least 10 theoretical stages or 16 actual stages. In an alternative embodiment, the light-ends column 130 may have at least 14 theoretical stages. In an alternative embodiment, the light-ends column 130 may have at least 18 theoretical stages. In embodiments, one actual stage may equal approximately 0.6 theoretical stages. Actual stages can be trays or packing. The reaction mixture may be fed via stream 126 to the light-ends column 130 at the bottom or the first stage of the column 130.

Overhead stream 132 may include acetaldehyde, water, carbon monoxide, carbon dioxide, methyl iodide, methyl acetate, methanol and acetic acid, one or more promoters and if present, catalyst stabilizer, and mixtures thereof. Bottoms stream 131 may have acetic acid, methyl iodide, methyl acetate, HI, water, and mixtures thereof. Stream 136 may have acetic acid, HI, water, heavy impurities, and mixtures thereof. Streams 132, 131, and 136, as well as other streams discussed herein, may also comprise catalyst stabilizers and/or promoters at varying concentrations, depending on where the catalyst stabilizers and/or promoters are added to the system.

In an embodiment, the light-ends column 130 may be operated at an overhead pressure within the range of 20 psia (138 kPa-a) to 40 psia (276 kPa-a), alternatively, the overhead pressure may be within the range of 30 psia (207 kPa-a) to 35 psia (241 kPa-a). In an embodiment, the overhead temperature may be within the range of 95° C. to 135° C., alternatively, the overhead temperature may be within the range of 110° C. to 135° C., alternatively, the overhead temperature may be within the range of 125° C. to 135° C. In an embodiment, the light-ends column 130 may be operated at a bottom pressure within the range of 25 psia (172 kPa-a) to 45 psia (310 kPa-a), alternatively, the bottom pressure may be within the range of 30 psia (207 kPa-a) to 40 psia (276 kPa-a).

In an embodiment, the bottom temperature of the light-ends column 130 may be within the range of 115° C. to 155° C., alternatively, the bottom temperature is within the range of 125° C. to 135° C. In an embodiment, crude acetic acid in stream 136 may be emitted from the light-ends column 130 as a liquid side-draw. Stream 136 may be operated at a pressure within the range of 25 psia (172 kPa-a) to 45 psia (310 kPa-a), alternatively, the pressure may be within the range of 30 psia (207 kPa-a) to 40 psia (276 kPa-a). In an embodiment, the temperature of stream 136 may be within the range of 110° C. to 140° C., alternatively, the temperature may be within the range of 125° C. to 135° C. Stream 136 may be taken between the fifth to the eighth actual stage of the light-ends column 130.

The overhead vapor in stream 132 from the light-ends column 130 may be condensed and separated in a decanter 134 to form a light, aqueous phase and a heavy, organic phase. The heavy, organic phase may be recycled to the reactor 110 in stream 138 via stream 112, for example. The stream 138 may comprise acetic acid, methanol, methyl acetate, methyl iodide, carbon monoxide, carbon dioxide, water, HI, heavy impurities, the one or more promoters, a catalyst stabilizer (optionally), and mixtures thereof.

The light, aqueous phase may be recycled to the light-ends column 130 in stream 135 or may be recycled to the reactor 110 in stream 139 via stream 112, for example. The stream 135 may comprise acetic acid, methanol, methyl acetate, methyl iodide, carbon monoxide, carbon dioxide, water, HI, heavy impurities, the one or more promoters, a catalyst stabilizer (optionally), and mixtures thereof. The heavy, organic phase in stream 138 may comprise methyl iodide, and methyl acetate, and mixtures thereof. The light, aqueous phase in streams 136 and 139 may comprise water (greater than 50%), acetic acid, comprise methanol, methyl acetate, methyl iodide, carbon monoxide, carbon dioxide, heavy impurities, the one or more promoters, a catalyst stabilizer (optionally), and mixtures thereof. Make-up water may be introduced into the decanter 134 via stream 133. Streams 139 and 138 may be considered to be in the light-ends area 104 and the recycle area 108.

In one or more embodiments, the crude acetic acid in stream 136 may be optionally subjected to further purification, such as, but not limited to, drying-distillation, such as in drying column 140 to remove water and heavy-ends distillation in stream 141. Stream 141 may be communicated to heavy-ends column 150 where heavy impurities such as propionic acid may be removed in stream 151 and final acetic acid product may be recovered in stream 156.

In an embodiment, the one or more promoters may be continually introduced and maintained into the system 100 via stream 160, and may be added in any form such as a solid, liquid, or solution, such as in an acetic acid solution. In FIG. 1, stream 160 mixes with the flash vessel 120 vapors to continually introduce the promoter to the components of the flash vessel 120 vapor stream 126. In an alternative embodiment, stream 160 may continually introduce the promoter into any equipment or streams in the reaction area 102, light-ends area 104, recycle arca 108, or combinations thereof. For example, stream 160 may continually introduce the promoter into the flash vessel 120, light-ends column 130, reactor 110, streams 111, 112, 114, 121, 126, 131, 132, 133, 135, 136, 138, 139, or combinations thereof. Therefore, although stream 160 is shown in FIG. 1 as mixing with the vapor stream emitted from the flash vessel 120, it is contemplated that alternative embodiments may include stream 160 as mixing with any equipment or stream in the reaction arca 102, light-ends area 104, recycle area 108, or combinations thereof.

In some embodiments, the one or more promoters disclosed herein may be continually introduced and maintained in stream 160 as a solution comprising the promoter and a solvent. In an embodiment, the promoter solution may comprise an acetic acid solution. The nature of the solvent or diluent generally may not be critical so long as the solvent or diluent does not interfere with the carbonylation reaction or the purification of the acetic acid in the purification arca 106.

Those skilled in the art of homogeneous processes having the benefit of this disclosure, and in particular those processes that require a flashing step to disengage involatile catalysts and promoter(s), will appreciate that attrition rates of catalysts and promoter(s) solely related to entrainment will be a function of several variables. Among these variables are reactor size, feed rate, flasher size, and flashing rate. They will also appreciate that a make-up solution of the one or more promoters disclosed herein in acetic acid could be as concentrated as the solubility limit of the promoters disclosed herein in acidic acid allows, which is about 50 wt % or as dilute as a few ppm.

A primary consideration is that the flow rate and concentration of the stream comprising the one or more promoters disclosed herein are matched such that there is a steady state concentration of one or more promoters in the reactor. This can normally vary from its high and low point between batch additions of up to 1.5 wt %, and in embodiments is controlled within a target range of ±0.5 wt % or in another embodiment ±0.2 wt %. Thus, for example in a process with an attrition rate of 1 wt % per month from the reactor, a monthly batch addition could be replaced by a continually metered stream corresponding to an average daily addition of the promoters disclosed herein of about 0.03 wt %.

In an embodiment, no solvent or diluent may be used. When a solvent or diluent is used in an embodiment, the solvent or diluent is one or more of the liquid constituents of the reaction mixture in reactor 110, e.g., acetic acid, methanol, methyl iodide, water, or combinations thereof. In an embodiment, the solvent or diluent may be acetic acid, methanol, or both. Similarly, the amount of solvent or diluents used in this context is not critical and may be adjusted broadly depending on process economy. The use of a solvent or diluent may be advantageous to ensure fast and even distribution and contact of the promoter with HI.

In an embodiment, when the one or more promoters is introduced to the system 100 separately and independently from the reaction mixture and from any recycle stream it may be advantageous to employ a solvent or diluent. Such a “recycle stream” may be a product or composition which is recovered from a processing step downstream of the flash vessel and which is recycled to the reactor, flash vessel, or light-ends column. In an alternative embodiment, when the one or more promoters is brought into contact with the reaction mixture in the flash vessel 120, for example, by adding it to stream 131 prior to introducing the stream 131 to the flash vessel 120, the promoters may be introduced in substance, i.e., in undiluted form, as the liquid constituents of the stream 131 act as solvents or diluents.

In any embodiment there is a greater than 5, or 10, or 16-fold rate increase in acetic acid production in the presence of (or, when combining) the one or more promoters disclosed herein. While not wishing to be bound by theory it believed that there is some synergistic interaction associated with the amino groups in the promoters. These groups likely quaternize with MeI to form the corresponding ammonium iodides leading to an electronic environment likely significantly different to the parent molecule that promotes oxidative addition with the carbonylation catalyst.

Further, while not wishing to be bound by theory, it is believed that as the amount of water in the reaction mixture is advantageously lowered HI will increasingly exist in the covalent H-I form rather than the dissociated H⁺—I⁻ or H₃O⁺—I⁻ form, and that the promoters drive that equilibrium towards the dissociated form. Primarily only the covalent form is capable of oxidative addition in which the following initial reaction occurs (C):

[Rh(CO)₂I₂)]⁻+2HI→[Rh(CO)₂I₄]⁻+H₂ (C)

The second part of the cycle involves interaction of the formed Rh III species with H₂O and CO (D):

[Rh(CO)₂I₄]⁻+H₂O+CO→[Rh(CO)₂I₂)]⁻+CO+2HI (D)

At low water concentration with HI predominantly in the covalent form, the rate limiting step is likely to be the second step shown above, whereas at high water concentration the first step is likely to be rate limiting. Thus, it is believed that the one or more promoters suppresses the oxidative addition of HI to the carbonylation catalyst.

In an embodiment, continually introducing one or more promoters may comprise continually or alternately metering the promoter(s) in solution (e.g., an acetic acid solution) using a gas or liquid metering technology known in the art such as turbine meters, Coriolis meters, ultrasonic meters, positive displacement meters, or combinations thereof. Continuously or alternately metering may comprise uniformly injecting a known concentration of promoter in solution (e.g., an acetic acid solution), or adjusting up and down the injection rate to suit a desirable need.

In an embodiment, the reaction mixture in reactor 110 does not comprise promoter other than the promoter continually introduced into the system 100 and which has been recycled to the reactor 110.

In an embodiment, the amount of promoter which is brought into contact with the HI, carbonylation catalyst, and/or water is generally not critical so long as the promoter is provided in an effective amount. An effective amount in this context is the amount of promoter which is capable of increasing the rate of producing acetic acid in the system 100. The amount of promoter that is added is preferably governed by the attrition rate of the promoter from the reactor rather than the HI concentration.

Generally, it is not detrimental to the subsequent separation and purification of the final acetic acid product if the molar amount of the one or more promoters exceeds the molar amount needed to promote the acetic acid reaction, so long as the boiling point of the promoter is sufficiently higher than the boiling point of the vapor stream 126 which emits from the flash vessel 120 and/or the stream 136 which emits from the light-ends column 130. For example, the boiling point of the promoter is sufficiently higher when the boiling point is at least 15° C., alternatively, at least 30° C., or alternatively, at least 50° C. above the boiling point of the crude acetic acid in stream 136.

In any embodiment the one or more promoters may be maintained in the flash vessel 120 in an amount from about 0.1 to about 1.5 mol per mol HI. In some embodiments, the amount of promoter is from about 0.1 to about 1.3 mol, or from about 0.1 to about 1.1 mol, per mol HI. In some embodiments, the amount of promoter is from about 0.5 to about 3 mol, or from about 0.5 to about 2 mol, or from about 0.5 to about 1.5 mol, per mol HI.

In further embodiments, the promoter may be maintained in the flash vessel 120 in an amount sufficient to establish a concentration of no more than about 20 wt % of the promoter in the liquid stream 121. In other embodiments, the promoter may be maintained to establish a concentration of no more than about 15 wt %, or no more than about 12 wt %, or no more than about 10 wt %, of the promoter in the liquid stream 121. In other embodiments, the promoter may be maintained in an amount sufficient to establish a concentration of at least about 0.5 wt % of the promoter in the liquid stream 121. In some embodiments, the promoter may be maintained in an amount sufficient to establish a concentration of at least about 1 wt %, or at least about 2.5 wt %, or at least about 4 wt %, of the promoter in the liquid stream 121. In particular embodiments, the promoter may be maintained in an amount sufficient to establish a concentration of from about 0.5 wt % to about 20 wt % of the promoter in the liquid stream 121.

In yet other embodiments, the promoter may be maintained in an amount sufficient to establish a concentration of from about 1 wt % to about 20 wt %, or from about 2.5 wt % to about 20 wt %, or from about 4 wt % to about 20 wt %, of the promoter in the liquid stream 121. In some embodiments, the promoter may be maintained in an amount sufficient to establish a concentration of from about 0.5 wt % to about 15 wt %, or from about 1 wt % to about 15 wt %, or from about 2.5 wt % to about 15 wt %, or from about 4 wt % to about 15 wt %, of the promoter in the liquid stream 121. In some embodiments, the one or more promoters may be maintained in an amount sufficient to establish a concentration of from about 0.5 wt % to about 12 wt %, or from about 1 wt % to about 12 wt %, or from about 2.5 wt % to about 12 wt %, or from about 4 wt % to about 12 wt %, of the promoter in the liquid stream 121.

The liquid stream 121 may be recycled to the reactor 110. The recycled liquid stream 121 may introduce the one or more promoters into the reactor 110, and consequently into the reaction mixture in reactor 110.

In some embodiments, the amount of the one or more promoters which is maintained in the flash vessel 120 may be adjusted to establish a steady state concentration of no more than about 20 wt % of the promoter in the reaction mixture. In alternative embodiments, the promoter may be maintained in the flash vessel 120 in an amount sufficient to establish a steady state concentration of no more than about 17 wt %, or no more than about 15 wt %, or no more than about 12 wt %, of the promoter in the reaction mixture. In other embodiments, the promoter may be maintained in the flash vessel 120 in an amount sufficient to establish a steady state concentration of at least about 2 wt % of the promoter in the reaction mixture. In alternative embodiments, the promoter may be maintained in the flash vessel 120 in an amount sufficient to establish a steady state concentration of at least about 5 wt %, or at least about 7 wt %, of the promoter in the reaction mixture.

In particular embodiments, the one or more promoters may be maintained in the flash vessel 120 in an amount sufficient to establish a steady state concentration from about 2 wt % to about 20 wt % of the promoter in the reaction mixture. In alternative embodiments, the promoter may be maintained in the flash vessel 120 in an amount sufficient to establish a steady state concentration of from about 5 wt % to about 20 wt %, or from about 7 wt % to about 20 wt %, of the promoter in the reaction mixture. In alternative embodiments, the promoter may be maintained in the flash vessel 120 in an amount sufficient to establish a steady state concentration of from about 2 wt % to about 17 wt %, or from about 5 wt % to about 17 wt %, or from about 7 wt % to about 17 wt %, of the promoter in the reaction mixture.

In some embodiments, the one or more promoters may be maintained in the flash vessel 120 in an amount sufficient to establish a steady state concentration of from about 2 wt % to about 15 wt %, or from about 5 wt % to about 15 wt %, or from about 7 wt % to about 15 wt %, of the promoter in the reaction mixture. In some embodiments, the promoter may be maintained in the flash vessel 120 in an amount sufficient to establish a steady state concentration of from about 2 wt % to about 12 wt %, or from about 5 wt % to about 12 wt %, or from about 7 wt % to about 12 wt %, of the promoter in the reaction mixture.

Residual HI which may reach the light-ends column 130 may be easily separated from as a bottom product stream 131 of the light-ends area 104. Also, due to the removal of HI from the product stream in the earliest stage of the acetic acid work-up, side reactions which are caused by HI, i.e., the formation of undesirable long chain alkyl iodide contaminants in the product stream downstream from the flash vessel, are significantly reduced. Additionally, the reduced amounts of HI in the product streams downstream from the flash vessel 120 alleviate corrosion and engineering problems.

The beneficial effect of the one or more promoters is not restricted to the point of introduction into the system 100. Rather, as the one or more promoters circulates in the system 100 by recycling the liquid stream 121 from the flash vessel 120 to the reactor 110, its presence in the reaction mixture aids promoting the rate limiting step in the acetic acid process and/or suppressing the reaction of HI with the carbonylation catalyst. Therefore, upon continuous operation of the process, the amount of promoter which is brought into contact with the reaction mixture in the flash vessel 120 normally may be decreased as steady state conditions are achieved. Under steady state conditions, the amount of promoter which is brought into contact with the reaction mixture in the flash vessel 120 normally can be reduced to amounts necessary to maintain the desired steady state concentration of the promoter.

Having described the various aspects of the inventive process, stated here in numbered paragraphs (P) is:
P1. A process for producing acetic acid comprising: a) combining methanol, methyl acetate or any mixture of the two, and carbon monoxide in the presence of a reaction mixture comprising: i) a carbonylation catalyst selected from the group consisting of rhodium catalysts, iridium catalysts, palladium catalysts, and cobalt catalysts; ii) water in the range of from 0.1 wt % to 10 wt %, based on the weight of the reaction mixture; and iii) one or more promoters selected from the group consisting of Group I and Group II polyphosphate and polyphosphonate salts and mixtures thereof; and iv) iodide; and b) recovering acetic acid.
P2. The process of numbered paragraph 1, wherein the polyphosphate and polyphosphonate salts comprise 2, or 3 or more phosphate or phosphonate groups.
P3. The process of numbered paragraphs 1 or 2, wherein the polyphosphate and polyphosphonate salts comprise 2, or 3 to 5, or 8, or 10 phosphate or phosphonate groups.
P4. The process of any one of numbered paragraphs 1 to 3, wherein the polyphosphate and polyphosphonate salts comprise at least one amine group and at least 3 phosphate or phosphonate groups.
P5. The process of any one of numbered paragraphs 1 to 4, wherein the polyphosphate and polyphosphonate salts comprise at least 2 amine groups and at least 2 phosphate or phosphonate groups.
P6. The process of any one of numbered paragraphs 1 to 5, wherein the iodide is in the form of HI, CH₃I, or both.
P7. The process of any one of numbered paragraphs 1 to 6, wherein the one or more promoters is continually added to the reaction mixture as an acetic acid solution.
P8. The process of any one of numbered paragraphs 1 to 6, wherein the process takes place in an acetic acid system comprising a reaction area, a light-ends area, a purification area, and a recycle area, wherein the reaction area comprises a reactor and a flash vessel.
P9. The process of numbered paragraph 8, wherein the one or more promoters are added to the flash vessel.
P10. The process of numbered paragraph 8, wherein the combining takes place in the reactor at a temperature within the range of about 120° C. to about 250° C. and a pressure within the range of about 200 psia (1.38 MPa-a) to 2,000 psia (13.8 MPa-a).
P11. The process of any one of numbered paragraphs 1 to 10, wherein the one or more promoters are maintained at an iodide to promoter molar ratio of 2:1 to 40:1.
P12. The process of any one of numbered paragraphs 1 to 11, wherein the one or more promoters are maintained at a promoter to carbonylation catalyst molar ratio of 0.005:1 to 4:1.
P13. The process of numbered paragraph 12, wherein as the molar ratio of the one or more promoters is increased the wt % of water can be decreased.
P14. The process of any one of numbered paragraphs 1 to 13, wherein salts of salts of glutamate and dicarboxymethyl alaninate are absent.
P15. A method for reducing water in an acetic acid production process comprising: a) combining methanol, methyl acetate or any mixture of the two, and carbon monoxide in the presence of a reaction mixture comprising iodide under carbonylation conditions sufficient to form acetic acid at a first rate, wherein the reaction mixture comprises: i) a carbonylation catalyst, selected from the group consisting of rhodium catalysts, iridium catalysts, palladium catalysts, and cobalt catalysts; and ii) a first amount of water, where the water is present in the range of from 0.1 wt % to 10 wt %, based on the weight of the reaction mixture; and b) combining one or more promoters to the reaction mixture at an iodide to promoter molar ratio of greater than 2, wherein the one or more promoters are selected from the group consisting of Group I and Group II polyphosphate and polyphosphonate salts and mixtures thereof; and c) reducing the wt % of water in the reaction mixture to a second amount of water while maintaining a second rate of acetic acid production that is the same as or greater than the first rate of acetic acid production.
P16. The process of numbered paragraph 15, wherein the polyphosphate and polyphosphonate salts comprise 2, or 3 or more phosphate or phosphonate groups.
P17. The process of numbered paragraphs 15 or 16, wherein the polyphosphate and polyphosphonate salts comprise 2, or 3 to 5, or 8, or 10 phosphate or phosphonate groups.
P18. The process of any one of numbered paragraphs 15 to 17, wherein the polyphosphate and polyphosphonate salts comprise at least one amine group and at least 3 phosphate or phosphonate groups.
P19. The process of any one of numbered paragraphs 15 to 18, wherein the polyphosphate and polyphosphonate salts comprise at least 2 amine groups and at least 2 phosphate or phosphonate groups.
P20. The process of any one of numbered paragraphs 15 to 19, wherein the iodide is in the form of HI, CH₃I, or both.
P21. The process of any one of numbered paragraphs 15 to 20, wherein the one or more promoters is continually added to the reaction mixture as an acetic acid solution.
P22. The process of any one of numbered paragraphs 15 to 21, wherein the process takes place in an acetic acid system comprising a reaction area, a light-ends area, a purification area, and a recycle area, wherein the reaction area comprises a reactor and a flash vessel.
P23. The process of numbered paragraph 22, wherein the one or more promoters are added to the flash vessel.
P24. The process of numbered paragraph 22, wherein the combining takes place in the reactor at a temperature within the range of about 120° C. to about 250° C. and a pressure within the range of about 200 psia (1.38 MPa-a) to 2,000 psia (13.8 MPa-a).
P25. The process of any one of numbered paragraphs 15 to 24, wherein the one or more promoters are maintained at an iodide to promoter molar ratio of 2:1 to 40:1.
P26. The process of any one of numbered paragraphs 15 to 25, wherein the one or more promoters are maintained at a promoter to carbonylation catalyst molar ratio of 0.005:1 to 4:1.
P27. The process of any one of numbered paragraphs 15 to 26, salts of glutamate and dicarboxymethyl alaninate are absent.

### EXAMPLES

The following investigations and examples are intended to be illustrative only, and are not intended to be, nor should they be construed as limiting the scope of the present invention in any way.

A 3,000 ppm stock solution of [Rh(CO)₂I₂]Li (often referred to simply as “Rh I”) in glacial acidic acid (GAA) was prepared as follows: 0.12 g of rhodium (I) dicarbonyl chloride dimer (CAS #14523-22-9) was added to 20 mLs of previously N₂ purged glacial acetic acid in a 30 mL vial. The vial was septum sealed and lightly purged with N₂ at one atmosphere while being stirred at room temperature. After 10 minutes, the septum was removed, 0.21 g of lithium iodide (LiI) was quickly added, the septum replaced and the mixture was stirred and purged for a further 10 minutes at which point full dissolution had occurred. The active Rh I catalyst has now formed and can be stored under an N₂ atmosphere indefinitely without decomposition. 100% formation of active catalyst was confirmed for each batch preparation by FTIR analysis of an aliquot. To start a kinetic run, 1.5 mLs of this stock solution was syringed into a stirred, septum sealed, N₂ purged vial containing 1.5 mLs of other components (promoter, MeI or HI, H₂O, glacial acidic acid). The starting Rh concentration is therefore, 1500 ppm or 0.015M. Typical conditions for MeI oxidative runs are shown in Table 1. The high concentration of MeI used ensures pseudo first-order conditions in all experiments and it is to be understood that the use of the term “rate constant” in text, figures and tables refers to pseudo-first order rate constants. Comparative conditions for similar experiments reported in U.S. Pat. No. 9,580,377 are also included in this Table 1.

TABLE 1
Conditions for Experiments

| Conditions | US ′377 | Inventive examples |
|---|---|---|
| Rhodium, ppm | 1200 | 1500 |
| H₂O, M | 1.3 | 1.3 |
| MeI, M | 1.6 | 2 |
| Atmosphere | N₂ | N₂ |
| Temp, ° C. | 22 | 22 |

Whenever possible, a constant H₂O concentration of about 1.3M (2 wt %) was maintained in kinetic experiments. For HI oxidative addition experiments, a concentration of 0.1M was chosen for most runs conducted. The 57% aqueous HI solution contributes 0.53M of H₂O to each kinetic experiment with the remainder, up to a total of 1.3M, being associated with H₂O content of concentrated promoter solutions. The various novel promoters that were investigated are listed in Table 2. The various fully aqueous and GAA/H₂O solutions that were prepared are shown in the table. In all cases, density measurements were made such that molarities could be calculated. All materials were soluble in H₂O at >40 wt % and in GAA/10 wt % H₂O at >20 wt %.

Note that the polyphosphate and polyphosphonate promoters were used as obtained so no stock solutions were prepared. The DTPMPA-Na₇ was an aqueous solution. The IP₆-Na₁₂ promoter was a solid and was weighed directly into vials; it was obtained as a hydrate with no quantitative information on water content. As knowledge of water content was useful to HI oxidative experiments, water in the commercial solid was first determined as described below. Table 3 is an example showing how IP₆-Na₁₂ solutions for kinetic runs were prepared and how water was calculated.

In particular, the determination of H₂O content of IP₆-Na₁₂ solid was performed as follows:
1. 0.141 g of IP₆-Na₁₂ was dissolved in 1.564 g of acetic acid;
2. A solution density of 1.115 g/mL was determined gravimetrically from the mass difference of an empty and full 250 μl syringe;
3. A total volume 1.513 mLs was then calculated; and
4. A transmission FTIR spectrum was then obtained and a water concentration of 2.29M was determined from a water FTIR calibration;
5. Which corresponds to 44 wt % water in original solid;
6. Therefore, the effective molecular weight of the hydrate was 1650 g/mole.

TABLE 2
Characteristics and Concentrations of Exemplary Promoters

| | MW, | | | | Wt % | Wt % |
|---|---|---|---|---|---|---|
| Promoter | g/mole | Wt % | M | Density | H₂O | GAA |
| DTPMPA-Na₇ | 727 | 42 | 0.76 | 1.308 | 58 | 0 |
| IP₆—Na₁₂ | 924 | NA | NA | NA | 44 | NA |

TABLE 3
Example of Calculations for IP₆-Na₁₂ Kinetic Experiments
3 mLs final volume for kinetic experiments:

| | | | | |
|---|---|---|---|---|
| Desired M | IP₆-Na₁₂ | 0.025 | 0.05 | 0.075 |
| Required Mass | Mass, g | 0.124 | 0.248 | 0.371 |
| Inherent H₂O, g | Mass H₂O | 0.05 | 0.11 | 0.16 |
| Inherent H₂O, M | M H₂O | 1.01 | 2.02 | 3.03 |
| Extra H₂O for 1.3M | Need H₂O, g | 0.015 | 0 | 0 |

FTIR spectroscopy was carried out as follows. An iS50 FTIR spectrometer equipped with a DTGS detector from Thermo Scientific was used for FTIR spectral acquisition. The sample compartment could optionally be fitted with a transmission cell accessory or with an attenuated total reflectance (ATR) accessory. The transmission cell, obtained from Harrick Scientific, was equipped with sapphire windows and was used with a pathlength of 0.1 mm. The ATR cell was obtained from Pike Technologies and was equipped with a 3-bounce zinc selenide (ZnSe) crystal. For the FTIR measurements in this disclosure, measurements of rhodium carbonyl bands in the 1950-2100 cm⁻¹ region were made and the two cells were largely interchangeable with regard to spectral acquisition time, quality of signal etc. The sample volume required for FTIR analysis was a function of cell being used. When the transmission cell was being used, a 1 ml syringe equipped with a needle was used to obtain about 0.3 mLs of sample from the septum sealed reaction vial (containing 3 mLs at outset). The sample was then loaded into the transmission cell, the cell was placed in the spectrometer sample compartment and a spectrum recorded. When the ATR cell was being used, the ATR accessory was fixed in the sample compartment and a much smaller sample volume (about 0.05 mLs obtained with a 100 μl microsyringe) was added to the ATR cell. For both cells, the sample was sealed to the atmosphere during spectral acquisition to avoid any trace air induced oxidation of the rhodium carbonyls.

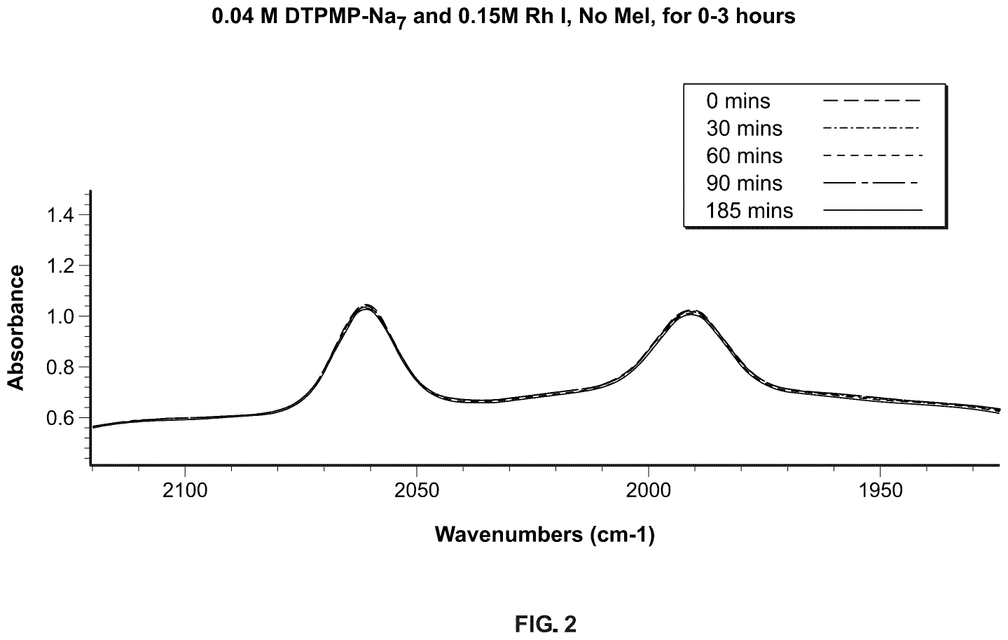

In a first blank experiment, all components required for an MeI oxidative addition experiment were assembled with the exception of MeI itself. In this experiment with 0.04M added DTPMP-Na₇, no direct reaction of the phosphonate with Rh I was observed over a 3 hour monitoring period as shown in the overlaid FTIR spectra in FIG. 2. Similar behavior was observed for a blank run with IP₆-Na₁₂. These observations indicate that spectroscopic changes that take place in experiments containing both MeI and rate accelerator are associated only with MeI interaction with Rh I.

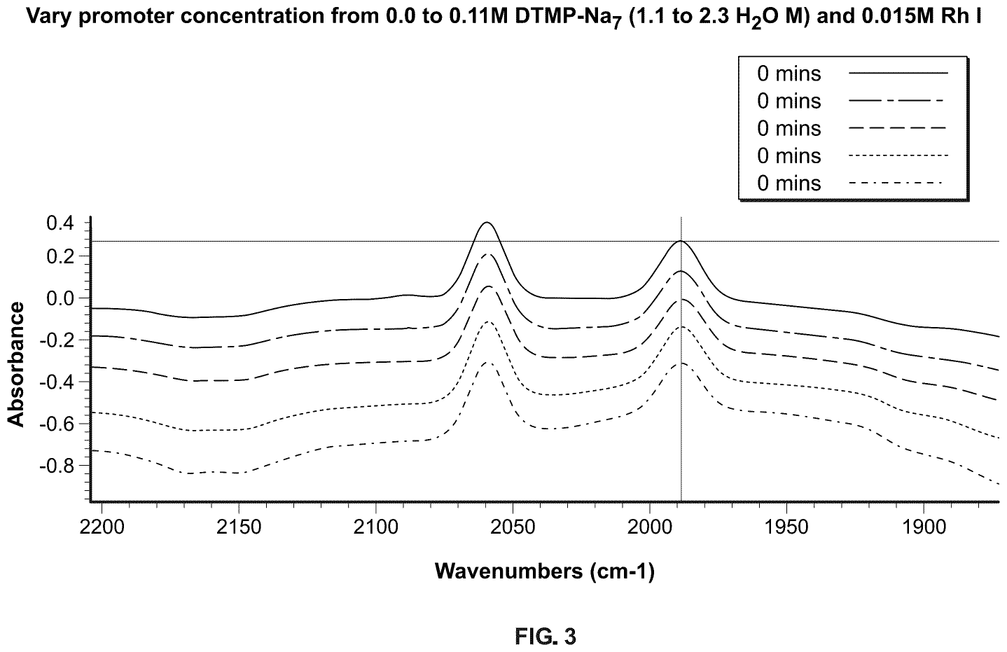

Overlaid FTIR spectra of variable concentration DTPMP-Na₇ solutions (0.0-0.11M) with Rh I show no evidence for any peak shifts as illustrated in FIG. 3 providing further evidence that no interaction is taking place with the Rh center.

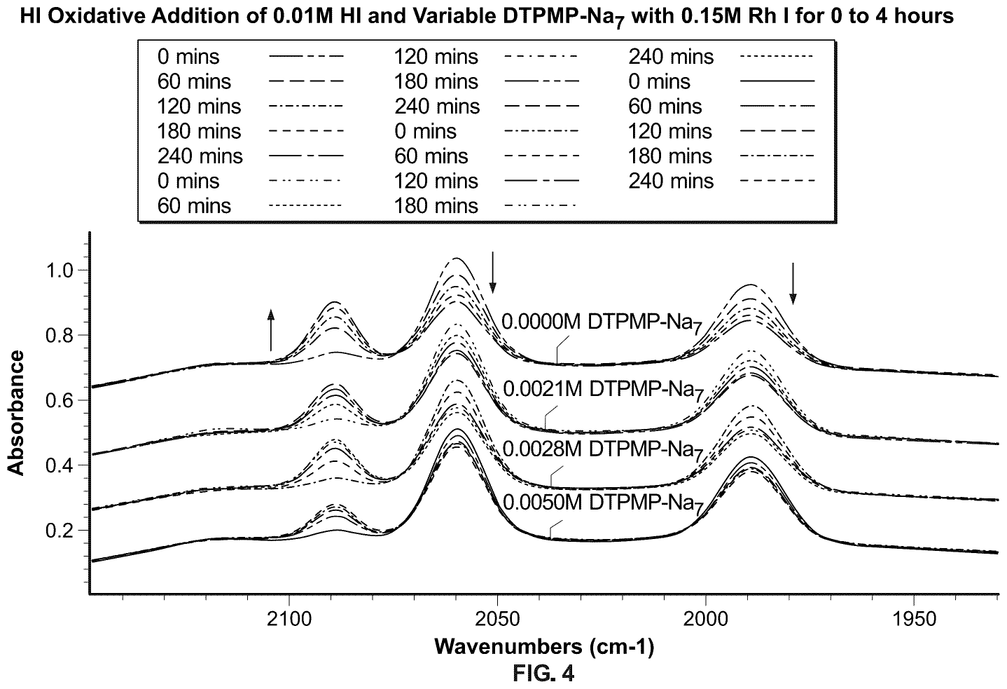

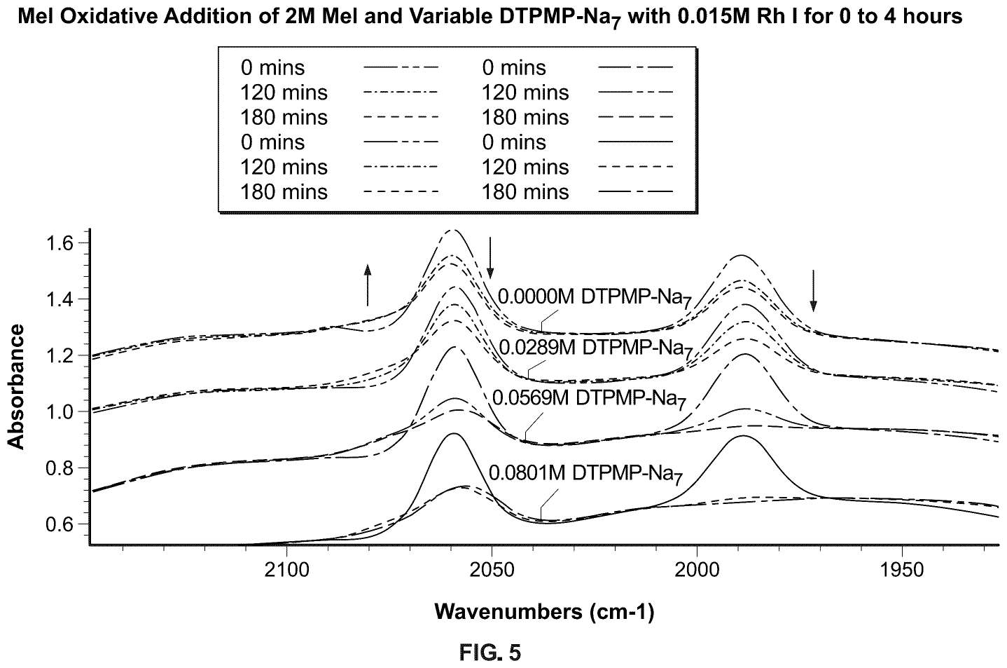

MeI and HI oxidative addition experiments with the novel polyphosphonate and polyphosphate promoters were carried out as described in the concurrently filed patent application titled “Aminopolycarboxylates as Rate Promoters for the Glacial Acetic Acid Process”. Representative overlaid FTIR spectra in FIG. 4 and FIG. 5 indicate that polyphosphonates also lead to unexpected significant promotion of MeI oxidative addition and to significant suppression of HI oxidative addition at very low concentration. Similar behavior was observed for corresponding experiments carried out with IP₆-Na₁₂.

Rate constant data for MeI oxidative addition in the presence of DTPMP-Na₇ presented in Table 4 show that a 12-fold increase in rate is achieved with only 0.1M of added DTPMP-Na₇. The initially determined rates are normalized to offset the slight rate increase associated with slightly increasing H₂O concentration n the commercial DTPMP-Naz aqueous solution. Corresponding MeI oxidative addition rate constants for IP₆-Na₁₂ are shown in Table 4 in which a similar large rate promotional effect associated with increasing concentrations of accelerator can be observed.

TABLE 4
MeI Oxidative Addition Rate Constants
with DTPMP-Na₇ Promoter

| M, DTPMP-Na₇ | k × 10³, min⁻¹ | k norm |
|---|---|---|
| 0 | 3.0 | 3.0 |
| 0.029 | 4.6 | 4.6 |
| 0.057 | 13.2 | 12.8 |
| 0.109 | 36.2 | 35.5 |

TABLE 5
MeI Oxidative Addition Rate Constants with
IP₆-Na₁₂ Promoter and corresponding acid

| | k × 10³, min⁻¹ | k norm |
|---|---|---|
| M, IP₆-Na₁₂ | | |
| 0 | 3.2 | 3.2 |
| 0.024 | 6.9 | 6.6 |
| 0.050 | 12.4 | 12 |
| 0.075 | 21.3 | 20.8 |
| M, IP₆ | | |
| 0.045 | 3.3 | 3.2 |
| 0.108 | 3.8 | 3.5 |

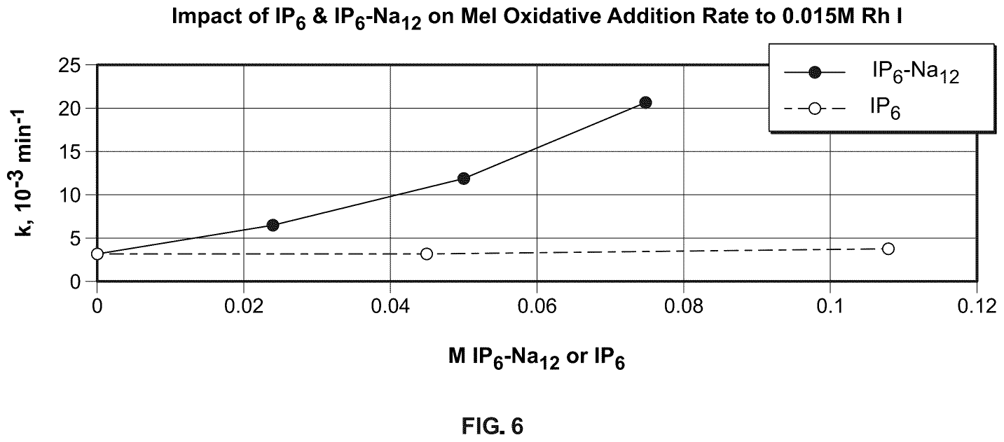

In addition, Table 5 also contains rate constants obtained with the parent phosphoric acid, IP₆, rather than the phosphate being added. In this case it can be observed that there is essentially no rate promotion. Information in Table 5 is presented graphically in FIG. 6. These data definitively demonstrate that there is no promotional effect associated with IP₆ over the same concentration range investigated for IP₆-Na₁₂.

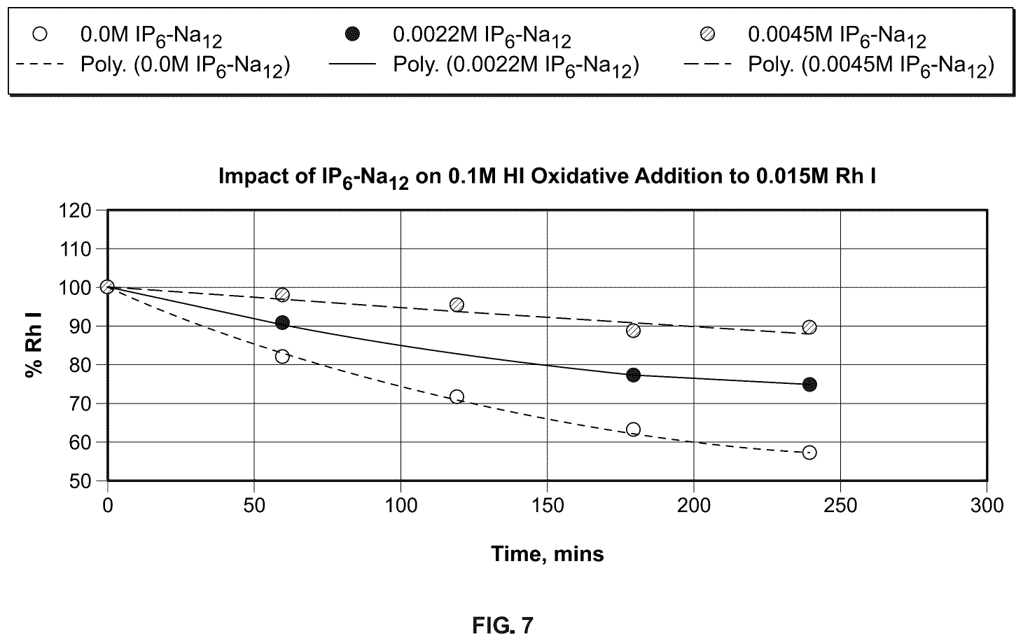

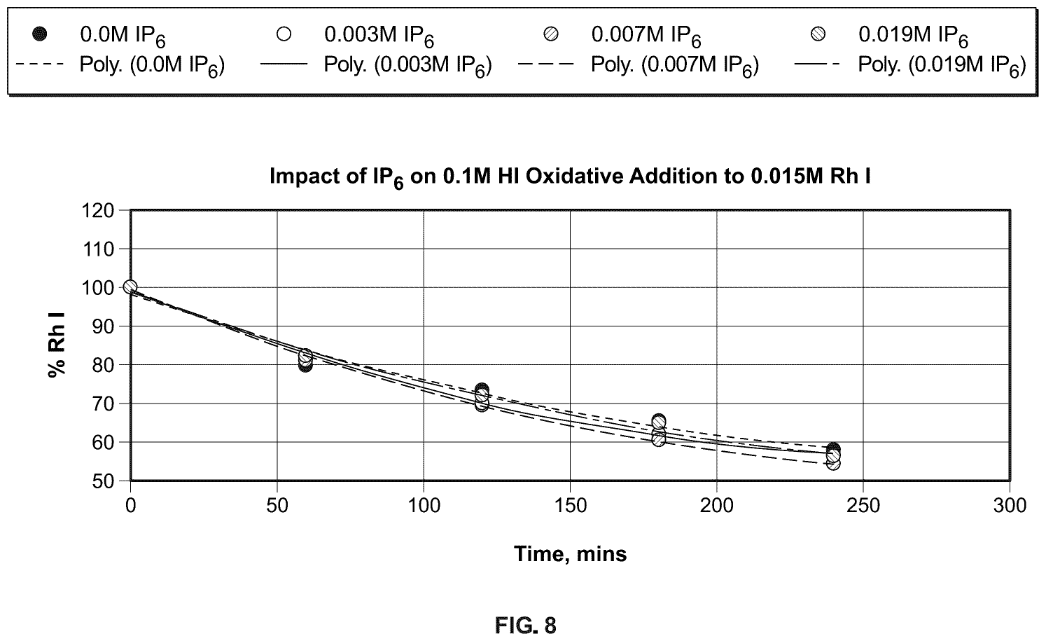

This requirement for a multiple salt functionality in lieu of the corresponding parent acid functionality for promotion of MeI oxidative addition was also demonstrated for suppression of HI oxidative addition. Time profiles for suppression of HI oxidative addition to Rh I at various IP₆-Na₁₂ concentrations are shown in FIG. 7 and at various IP₆ concentrations in FIG. 8. The data show that the protection of Rh I provided by IP₆-Na₁₂ is entirely absent when only the parent acid, IP₆ is present.

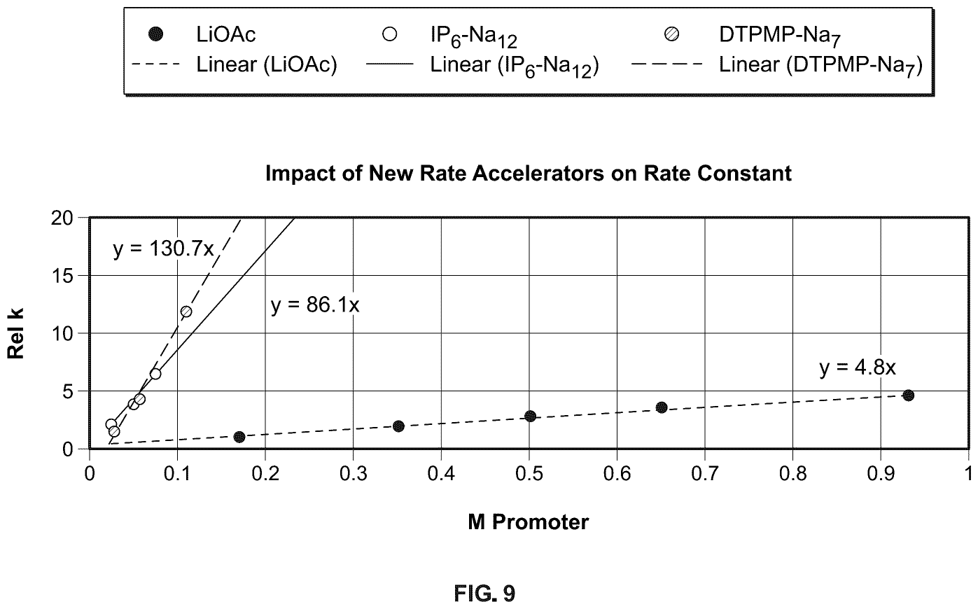

Rate promotional data associated with DTPMP-Na₇ and IP₆-Na₁₂ are plotted along with previously obtained data for LiOAc in FIG. 9. The slopes of these plots are presented as rate constants (k values) in Table 6 relative to the slope of the LiOAc plot which is assigned a value of 1. The included molecular weight data show that even allowing for the high MW of these new rate accelerators, their large effect at low concentration still gives them an advantage with regard to required mass.

TABLE 6
Comparison relative rate constants for LiOAc and Novel Promoters

| Accelerator | # Salt Groups | Rel k | MW | Rel MW |
|---|---|---|---|---|
| LiOAc | 1 | 1 | 66 | 1 |
| DTPMP-Na₇ | 7 | 27.2 | 727 | 11 |
| IP₆-Na₁₂ | 12 | 17.9 | 923 | 14 |

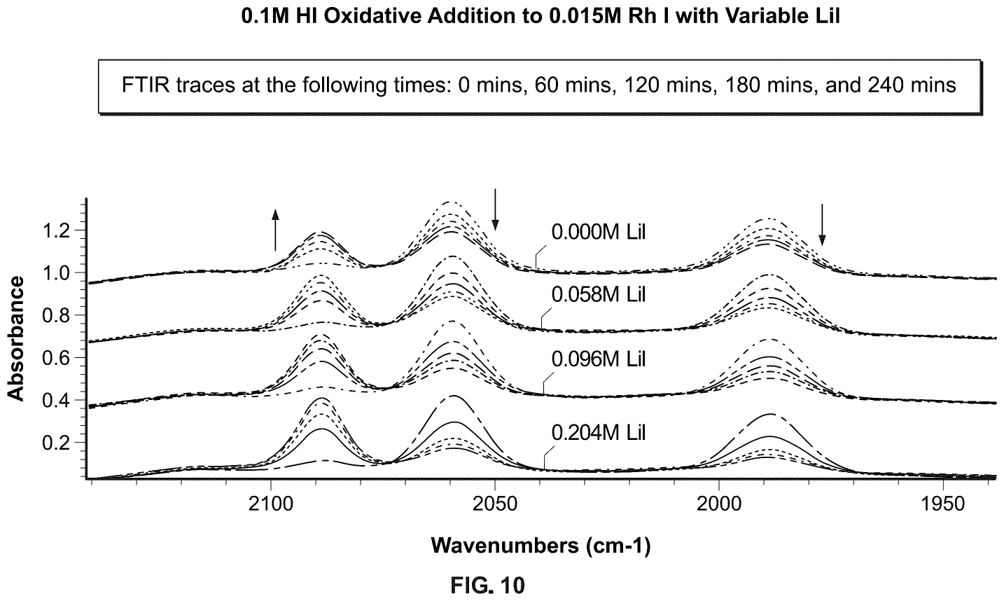

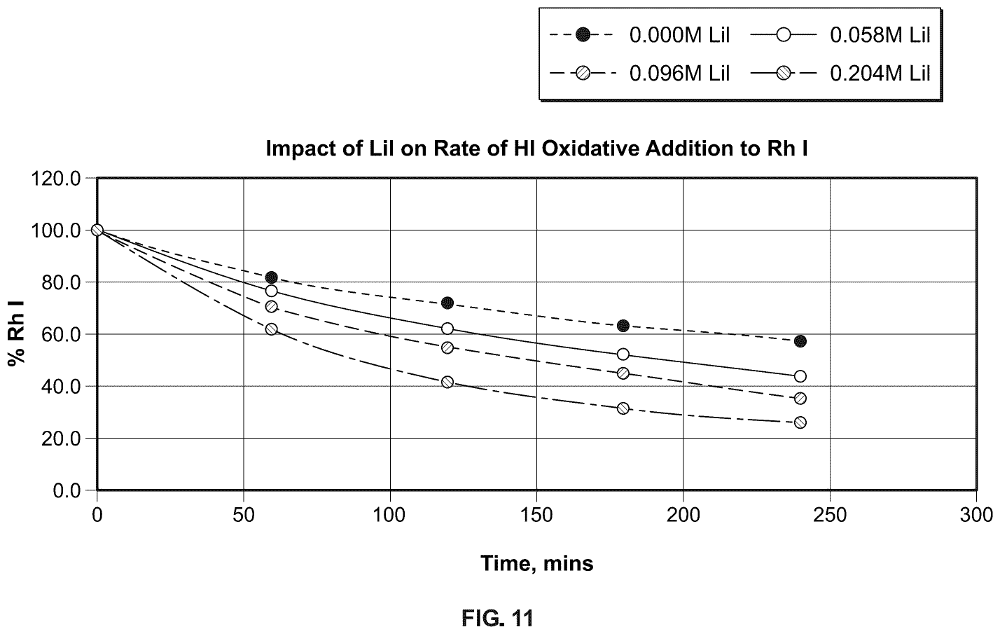

Also investigated were the kinetics associated with the effect of LiI on HI oxidative addition rate. It has been surprisingly found that LiI, rather than suppressing HI oxidative addition, does the reverse and leads to acceleration. This is shown in the four overlaid sets of FTIR spectra in FIG. 10 in which increasing LiI concentration leads to increased extent of HI oxidative addition to Rh I over the four hour monitoring period. When a blank experiment is carried out, 0.2M LiI but no HI, no changes in to the initial spectrum are observed over four hours. Quantitative measurements associated with the spectra in FIG. 10 were used to construct the time profiles of Rh I decay as shown in FIG. 11.

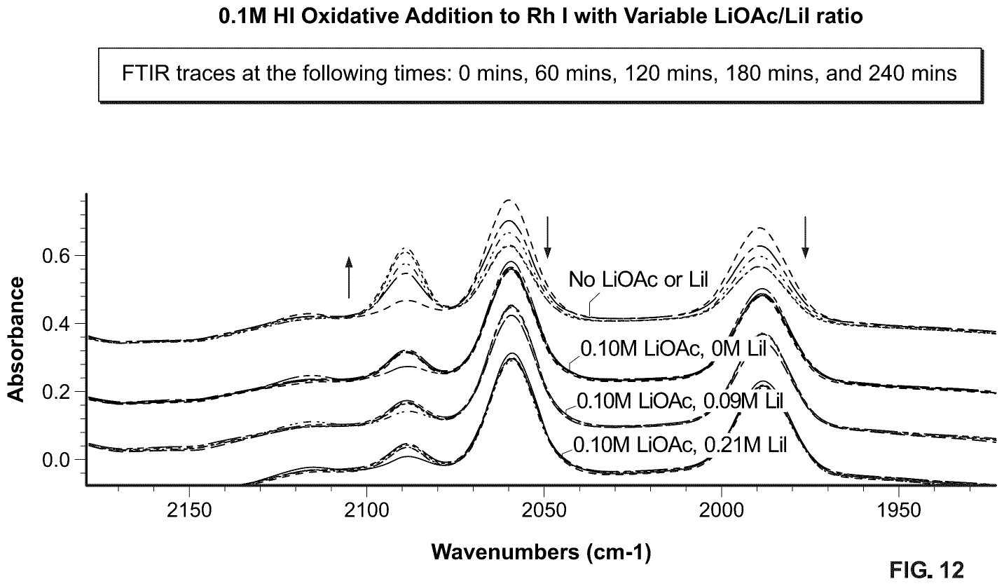

The data above demonstrate that LiI does not suppress HI oxidative addition. This finding prompted further experiments to determine the effect of a LiI/LiOAc mixture on both MeI oxidative addition and HI oxidative addition. FTIR overlay data associated with HI oxidative addition on variable LiI/LiOAc mixture are shown in FIG. 12. The second from top set of spectra show the previously demonstrated behavior where a molar equivalence of LiOAc relative to HI essentially shuts down HI oxidative addition. The two bottom sets of spectra show that in the presence of both LiI and LiOAc, the undesired LiI effect observed in FIG. 10 is no longer present and the LiOAc desired effect on HI oxidative addition suppression dominates. These new findings suggest that in any LiI/LiOAc mixture, LiOAc is the “heavy lifter” with regard to suppression of HI oxidative addition.

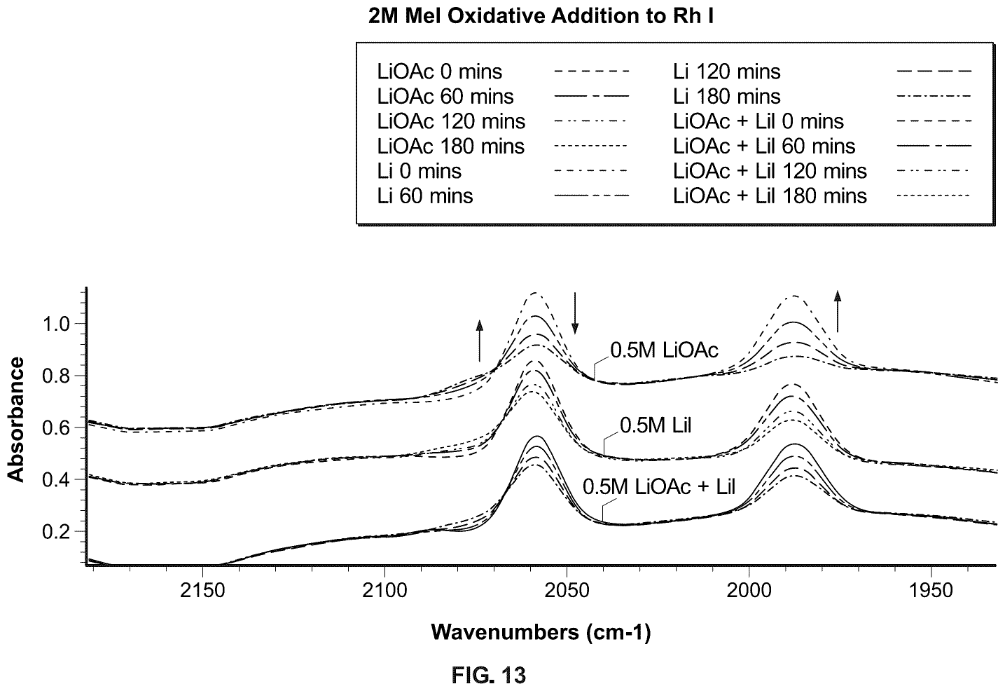

The corresponding study with regard to MeI oxidative addition led to a further surprising finding. As the overlaid spectra in FIG. 13 visually indicate, the lower rate promotional effect of LiI alone compared to LiOAc alone is reproduced in an equimolar mixture. That is to say, the promotional effect of LiI dominates in a mixture which is the reverse of the trend observed for suppression of HI oxidative addition.

Principal data from a concurrently filed patent application titled “Aminopolycarboxylates as Rate Promoters for the Glacial Acetic Acid Process”, along with data from this disclosure are summarized in Table 7 in which two indices have been devised. A Carbonylation Reactivity Index (CRI) and a Shift Suppression Index (SSI) are associated with MeI oxidative addition and HI oxidative addition, respectively. In both cases the extent of promotion or suppression is expressed proportionally relative to promotion and suppression observed for LiOAc with both values being assigned a value of 1. The negative highlighted values for Cytop™ 503/MeI oxidative addition and for LiI/HI oxidative addition indicate that in these two cases the observed effects are directionally opposite to all other tested compounds. It can be noted that for any new promoter other than IP₆, where data are available for both columns, a directionally desired larger effect than obtained for LiOAc is obtained for both columns.

TABLE 7
Summary Table of Novel Oxidative
Addition Rate Promoter Compounds

| | 2M MeI | 0.1M HI |
|---|---|---|
| Promoter | CRI | SSI |
| LiOAc | 1 | 1 |
| LiI | 0.11 | −0.95 |
| LiI/LiOAc | 0.11 | 1 |
| Cytop ™ 503 | −0.11 | 2.2 |
| NTA-Na₃ | 5 | 5 |
| GLDA-Na₄ | 7.3 | 7.7 |
| DTPA-Na₅ | 17.3 | 11 |
| DTPMP-Na₇ | 27.2 | 14.2 |
| IP₆-Na₁₂ | 17.9 | 20 |
| IP₆ | 0 | 0 |

CRI = Carbonylation Reactivity Index
SSI = Shift Suppression Index

While not wishing to be bound by theory, it is postulated that there are at least two properties of these new rate accelerators that lead to their ability to induce a novel and unexpected increase in MeI oxidative addition rate. The first of these is the high intramolecular concentration of salt groups (phosphonates or phosphates) which are not present in salts such as LiI and LiOAc. In these new promoters, salt group proximity coupled with their known chelating ability may play a role in their rate promotional effect. The second property of these promoters is associated with the presence, in most, of an amino backbone. In a GAA medium rich in iodide (MeI or HI), quaternization to the corresponding ammonium acetate, ammonium iodide or a mixture of both will almost certainly take place, even at room temperature. The new amino-containing promoters introduce a dual intramolecular mixed salt functionality which is concurrently filed as a patent application entitled “Aminopolycarboxylates as Rate Promoters for the Glacial Acetic Acid Process” postulated as having potential for superior promotional affect over the intermolecularly regulated LiI/LiOAc combination.

Some evidence for the above theories comes from the finding that while DTPMP-Na₇ leads to a 30-fold rate promotional effect, IP₆-Na₁₂ has only a 20-fold promotional effect. Based on the number of salt groups in IP₆-Na₁₂, it might be expected to have a substantially higher promotional effect than DTPMP-Na₇. However, IP₆-Na₁₂ lacks an amino backbone which may explain its lower than expected activity.

The scope of the present application is not intended to be limited to the particular embodiments of the promoters, processes, means, methods, and/or steps described in the specification. The particular embodiments disclosed above are illustrative only, as the process and system may be modified and practiced in different but equivalent manners apparent to those skilled in the art having the benefit of the teachings herein.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All patents, test procedures, and other documents cited in this application are fully incorporated herein by reference for all jurisdictions in which such incorporation is permitted. In the event of conflict between one or more of the incorporated patents or publications and the present disclosure, the present specification, including definitions, controls.

What is claimed is:

1. A process for producing acetic acid comprising:
a) combining methanol methyl acetate or any mixture of the two, and carbon monoxide in the presence of a reaction mixture comprising:
i) a carbonylation catalyst selected from the group consisting of rhodium catalysts, iridium catalysts, palladium catalysts, and cobalt catalysts;
ii) water in the range of from 0.1 wt % to 10 wt %, based on the weight of the reaction mixture; and
iii) one or more promoters selected from the group consisting of Group I and Group II polyphosphate and polyphosphonate salts and mixtures thereof; and
iv) iodide; and
b) recovering acetic acid.

2. The process of claim 1, wherein the iodide is in the form of HI, CH₃I, or both.

3. The process of claim 1, wherein the one or more promoters is continually added to the reaction mixture as an acetic acid solution.

4. The process of claim 1, wherein the process takes place in an acetic acid system comprising a reaction area, a light-ends area, a purification area, and a recycle area, wherein the reaction area comprises a reactor and a flash vessel.

5. The process of claim 4, wherein the one or more promoters are added to the flash vessel.

6. The process of claim 1, wherein the one or more promoters are maintained at an iodide to promoter molar ratio of 2:1 to 40:1.

7. The process of claim 1, wherein the one or more promoters are maintained at a promoter to carbonylation catalyst molar ratio of 0.005:1 to 4:1.

8. The process of claim 7, wherein as the molar ratio of the one or more promoters is increased the wt % of water can be decreased.

9. The process of claim 1, wherein salts of salts of glutamate and dicarboxymethyl alaninate are absent.

10. A method for reducing water in an acetic acid production process comprising:
a) combining methanol, methyl acetate or any mixture of the two, and carbon monoxide in the presence of a reaction mixture comprising iodide under carbonylation conditions sufficient to form acetic acid at a first rate, wherein the reaction mixture comprises:
i) a carbonylation catalyst, selected from the group consisting of rhodium catalysts, iridium catalysts, palladium catalysts, and cobalt catalysts; and
ii) a first amount of water, where the water is present in the range of from 0.1 wt % to 10 wt %, based on the weight of the reaction mixture; and
b) combining one or more promoters to the reaction mixture at an iodide to promoter molar ratio of greater than 2, wherein the one or more promoters are selected from the group consisting of Group I and Group II polyphosphate and polyphosphonate salts and mixtures thereof; and
c) reducing the wt % of water in the reaction mixture to a second amount of water while maintaining a second rate of acetic acid production that is the same as or greater than the first rate of acetic acid production.

11. The process of claim 10, wherein the iodide is in the form of HI, CH₃I, or both.

12. The process of claim 10, wherein the one or more promoters is continually added to the reaction mixture as an acetic acid solution.

13. The process of claim 10, wherein the process takes place in an acetic acid system comprising a reaction area, a light-ends area, a purification area, and a recycle area, wherein the reaction area comprises a reactor and a flash vessel.

14. The process of claim 13, wherein the one or more promoters are added to the flash vessel.

15. The process of claim 10, wherein the one or more promoters are maintained at an iodide to promoter molar ratio of 2:1 to 40:1.

16. The process of claim 10, wherein the one or more promoters are maintained at a promoter to carbonylation catalyst molar ratio of 0.005:1 to 4:1.

17. The process of claim 10, salts of glutamate and dicarboxymethyl alaninate are absent.
